(19) United States
(12) Patent Application Publication
(10) Pub. No.: US 2009/0074139 A1
(43) Pub. Date: Mar. 19, 2009

(54) Method and an apparatus for detecting and localizing a metabolic marker

(76) Inventors: Eckhard Hempel, Furth (DE); Martin Hoheisel, Erlangen (DE); Stefan Popescu, Erlangen (DE); Rainer Raupach, Heroldsbach (DE)

Correspondence Address: HARNESS, DICKEY & PIERCE, P.L.C., P.O.BOX 8910, RESTON, VA, 20195, US

(21) Appl. No.: 12/222,077

(22) Filed: Aug. 1, 2008

(30) Foreign Application Priority Data
Aug. 3, 2007 (DE) 10 2007 036 559.6

(51) Int. Cl. G01N 23/04; A61B 6/03

(52) U.S. Cl. 378/62; 600/407

(57) ABSTRACT

A method and an x-ray system are disclosed for detecting and localizing a metabolic marker. In at least one embodiment, the method includes creating at least one absorption x-ray view of a patient or of a first region of a patient; creating at least one phase-contrast x-ray view of the patient or of a second region of the patient, with a quasi-coherent x-ray radiation being generated for the phase-contrast measurement with the aid of an x-ray grating arranged between the x-ray source and the patient, and the spatially dependent phase shift of the x-ray radiation in the patient being made visible with the aid of at least one grating between the patient and a detector; superposing the at least one absorption x-ray view and the at least one phase-contrast x-ray view; wherein orientation based on anatomical features is carried out with the aid of the at least one absorption x-ray view, and a spatial distribution of the metabolic marker present in the body of the patient is determined by the at least one phase-contrast x-ray view.

### PRIORITY STATEMENT

The present application hereby claims priority under 35 U.S.C. §119 on German patent application number DE 10 2007 036 559.6 filed Aug. 3, 2007, the entire contents of which is hereby incorporated herein by reference.

### FIELD

Embodiments of the invention generally relate to a method and/or an apparatus for detecting and localizing a metabolic marker in a patient using a combination of an absorption x-ray examination and a phase-contrast x-ray examination.

In at least one embodiment, a functional or molecular imaging system and imaging method are intended to be presented which calculate additional data about the biological functions of an organ in a living body. In at least one embodiment in more detail, they are not only intended to create more precise images using the new method and the new appliance, which images give information about inner anatomical details of the body, as is already known from published CT or MRI imaging methods, but rather they are intended to also obtain corresponding information about the metabolism of a number of organs.

### BACKGROUND

Information can be obtained by way of PET and SPECT, as described in “The Power of Molecular Imaging” by Jennifer Jones Simon Foundation, UCLA School of Medicine, Institute for Clinical PET and U.S. Department of Energy. These are medical imaging methods which produce images of the biological functions of the human body and which can remove ambiguities in the case of cancers. In the case of these examinations, simple compounds such as glucose are labeled with radioactive markers and injected into the human body. A scanner receives the signals which the marker emits on its path through the human body, as glucose is distributed and accumulates in various organs. A computer reconstructs these signals to form images, which can give information about the normal biological functions or the malfunctions of some organs. This is possible because, although glucose is used by all cells, it is more intensively used by cells with an increased metabolism. Cancerous cells have a greatly increased metabolism and use up more glucose than healthy cells; thus the glucose metabolism products accumulate in increased amounts in these cells, and the attached markers can easily be localized in a PET/SPECT scan. With this knowledge, the medical practitioner can determine the best treatment method or check its effectiveness after cancer treatment using a follow-up scan.

Furthermore, the PET method is currently the most accurate possibility to demonstrate or rule out coronary diseases. By way of PET/SPECT images, an inadequate blood flow to the heart in stress situations can be recognized; this is not recognized by other non-invasive cardiac examinations. If these scans demonstrate that the blood flow and the metabolism connected therewith are missing in a large region of the heart, this is a reliable indication that this part of the myocardium has died off as a result of an aggressive myocardial infarction, that is to say irreversible necrosis has occurred. If the images demonstrate only a reduced metabolism, this is an indication of a severe ischemic process, that is to say a muscular lesion, and the medical practitioner can determine the further treatment method, for example a bypass, for this reversible pathology.

A further possible application of the PET scan is the regular diagnosis patterns in Alzheimer's disease, in which particular regions of the brain have a decreased metabolism. This even occurs a few years before a medical practitioner can provide a diagnosis. Additionally, the metabolic scan helps in differentiating Alzheimer's disease from other complex forms of dementia or depression. Images of the molecular metabolism of the brain identify regions with a reduced glucose metabolism which indicate epileptogenic tissue and can successfully be removed by surgery. Even Parkinson's disease can be discovered by means of PET images of the brain, if an injection marked with amino acids which uncover a deficit of dopamine synthesis has been administered to the brain.

Even though these molecular imaging methods are very useful for functional medical diagnostics, PET and SPECT scanners are expensive and difficult to operate. All positron emitters used as markers have a short life. In order to make a marker clinically useful, the transport time must be shorter than its half-life. Only ¹⁸F, with a half-life of 110 minutes, can be transported over distances of up to 200 kilometers, and then only for immediate subsequent use. Other positron emitters must be produced in powerful cyclotrons in the hospital or at least in close proximity to the intended point of use. In an efficient manner, one such installation supplies a number of hospitals with many patients to be treated every day.

Furthermore, the spatial resolution of these molecular imaging methods is problematic. It is very low compared to the resolutions of anatomical scanners such as CT or MRI. In fact, it is so coarse-grained that the anatomical details can be recognized only with great difficulty in the PET/SPECT images. In order to overcome this problem, the prior art uses a highly developed and very expensive mix of the appliances, comprising a combined CT and PET scanner, a combined CT and SPECT scanner or a combined MRI and PET scanner. By means of image fusion, this method mixes the superior anatomical resolution of the CT/MR images with the functional information of the molecular images from the PET/SPECT scanners.

A further disadvantage of these known PET/SPECT imaging methods is the very long scan time required to obtain initial data. In general, this takes 20-30 minutes, depending on the scanning increment. Because of this, these images also tend to have movement artifacts.

### SUMMARY

In at least one embodiment of the invention, an alternative, more cost effective scanning method is disclosed, which has a higher resolution and is faster, and a better scanning apparatus which can likewise demonstrate molecular processes in the body of a patient.

The inventors have recognized, in at least one embodiment, that it is possible to demonstrate changes in the concentration of metabolic substances on a molecular level with the aid of a phase-contrast x-ray CT system, with the spatial resolution being much higher than in the case of PET systems or SPECT systems. Furthermore, it is possible to simultaneously obtain data for phase-contrast imaging and absorption imaging via a detector by means of a phase-contrast x-ray CT scan. With the aid of the absorption data, very good localization of anatomical conditions in the patient is possible, while the phase-contrast data can give information about the concentration of metabolic substances. As a result of this, it is no longer necessary to administer radioactive products into the body of the patient, which can locally result in a high probability of cell damage or malign changes in cells. In at least one embodiment of the present method, “metabolic markers” are thus used which occur in the natural metabolism of a patient, without radioactive markings being necessary.

Thus, according to at least one embodiment of this new solution, at least one phase-contrast scan and at least one absorption scan are carried out, and both can be able to be carried out by a single detector scan. Subsequently:
1. anatomical images with a high spatial resolution, comparable to the prior art using the CT method, and
2. functional images for examining the cell metabolism are reconstructed.

In contrast to previous PET-CT methods, which combine simple sugar with radioactive markers in order to track the accumulation of glucose in organs, the new method detects these metabolic markers, such as glucose, in a natural manner. This method, in at least one embodiment, is possible because x-ray phase-contrast imaging has much higher contrast sensitivity than the classical method of x-ray absorption imaging. By way of example, “Vessel Imaging by Interferometric Phase-Contrast X-Ray Technique”, T. Takeda, Circulation 2002, 105:1708-1712, has already disclosed that x-ray phase-contrast imaging can even detect small concentrations of glucose (less than 5%) in pure water or make blood vessels visible using only small doses of x-ray radiation using a simple physiological common saline solution as a contrast agent. However, the method of x-ray phase-contrast imaging disclosed there is based on a crystal interferometric method, also known as Bonse & Hart interferometry, and it has a number of disadvantages making it impractical for imaging large objects, for example the human body:
1. It requires monochromatic x-ray radiation, which is not available with a sufficient signal quality at the intensity required for large bodies in the case of suitably short scan times.
2. The Bonse & Hart interferometer cannot be produced in a size corresponding to the dimensions of the human body, or it is not sufficiently mechanically robust with these dimensions.
3. This method generates only images of the gradient of the phase.

By contrast, the new solution presented by the inventors uses an arrangement to measure the phase-contrast based on the Talbot interferometry method and has these characteristics, described in the following:
1. It operates using conventional polychromatic x-ray tubes.
2. It can also be used for large bodies.
3. It creates not only images of the gradient of the phase, but also conventional absorption images using the same scanning technique without an additional x-ray dose.

According to a second aspect of at least one embodiment, the inventors have combined the newly developed method with the method of digital image subtraction. In the process, a first reference scan is carried out prior to the metabolic marker being injected. Subsequently, a second scan is carried out as soon as it is assumed that the accumulation of the metabolic marker within the target organ is maximal. Better separation of the metabolic marker in target organ is achieved by image registration and image subtraction.

The method of at least one embodiment described above is however not limited only to CT views. Corresponding evaluations are also possible having only 2-dimensional spatial information using projection recordings.

Based on these fundamental ideas, the inventors propose a method for, in at least one embodiment, detecting and localizing a metabolic marker which comprises:

creating at least one absorption x-ray view of a patient or of a first region of a patient,

creating at least one phase-contrast x-ray view of a patient or of a second. region of a patient, with the second region lying in the area of the first region and a quasi-coherent x-ray radiation being generated for the phase-contrast measurement with the aid of an x-ray grating arranged between the x-ray source and the patient, and the spatially-dependent phase shift of the x-ray radiation in the patient being made visible with the aid of at least one grating between the patient and a detector,

superposing the at least one absorption x-ray view and the at least one phase-contrast x-ray view,

with an anatomical orientation being carried out with the aid of the at least one absorption x-ray view and the recognition of the spatial distribution of a metabolic marker present in the body of the patient being carried out by the at least one phase-contrast x-ray view.

It is pointed out that “metabolic markers” are not understood to be radioactively marked substances, that is to say substances which themselves actively emit radiation, but are substances which are functionally integrated into the metabolic system of the body, cause a significant phase shift and show medically relevant states and locations due to a difference in the strength of the concentration.

In specific cases, it may be advantageous for the patient to be administered the observed metabolic marker prior to the creation of at least one absorption x-ray view and at least one phase-contrast x-ray view.

If a first scan to create at least a first absorption x-ray view and at least a first phase-contrast x-ray view is carried out prior to a dose of the metabolic marker being administered and if, after the dose of the metabolic marker has been administered, a second scan to create at least a second phase-contrast x-ray view is carried out, then locations of increased accumulations of the markers can be determined particularly well, even if the natural metabolism is deficient with regard to the markers. In this context, the second scan can additionally also be carried out to create a second absorption x-ray view.

Furthermore, for the purpose of improved visual display of the difference prior to and after administering a marker, it can be advantageous if a difference image between the first and second phase-contrast x-ray view is created and displayed in order to accentuate the applied metabolic marker.

In order to obtain an optimum anatomical orientation, one of the absorption x-ray views can also be superposed on the difference image.

Since a certain amount of time elapses between the first and the second scan, and a patient or his organs cannot be fixed in a reliable manner, it is particularly advantageous if the created records are spatially related to one another or are spatially normalized, with the patient himself in each case forming the reference coordinate system. For this purpose, it is suggested that the first phase-contrast x-ray view is registered to the first absorption x-ray view, the second phase-contrast x-ray view is registered to the second absorption x-ray view, and two spatially corrected phase-contrast x-ray views are used to create a spatially corrected difference image, with the difference image being used to accentuate the applied metabolic marker.

Additionally, a corresponding or spatially corrected absorption x-ray view can also be superposed on the spatially corrected difference image. Particularly good recognition of even small measured value differences can be achieved if the phase-contrast x-ray view or the phase-contrast difference views and the absorption x-ray view are displayed in different colors. In this context, there are a multiplicity of different variants. By way of example, the absorption view can be displayed in black and white in a conventional manner and the phase differences can be displayed in color, or vice versa. Different color combinations can likewise be selected or the intensity of a measured value can be correlated to the spectral frequency.

When administering an additional substance, a period of time should be allowed to elapse between the metabolic marker being administered and the second scan, before an expected maximal marker concentration can be expected at the observed location.

If the abovementioned interim period is not known exactly, or is too dependent on individual physiological parameters of the patient being examined, it is suggested that intermediate scans be carried out with a reduced dose between the metabolic marker being administered and the second scan, which scans reveal when a maximal marker concentration has been reached in a region of interest.

With regard to the relationship between the scanning regions, the first and the second region can, on the one hand, be identical. However, on the other hand, it is also possible—for cost considerations due to the complex x-ray gratings, for example—for the first region to have a larger extent than the second region.

Depending on the purpose, different substances can be chosen as metabolic markers. By way of example, the use of glucose can be advantageous, in which case the occurrence of an increased glucose concentration or an increased glucose-6-phosphate concentration relative to the rest of an organ or the scan surroundings can be interpreted as an increased probability of the presence of a tumor.

By way of example, NaCl (common salt) can be a further metabolic marker. This allows the vascularization in the region of tumoral tissue to be displayed particularly well.

According to at least one embodiment of the invention, the method described above can, on the one hand, be used combined with an x-ray CT system, so that an absorption x-ray CT view is reconstructed and used as an absorption x-ray view, and a phase-contrast x-ray CT view is reconstructed and used as a phase-contrast x-ray view.

Alternatively, in at least one embodiment, it is also possible to use this method in purely projective imaging, so that an absorption x-ray projection view is used as an absorption x-ray view and a phase-contrast x-ray projection view is used as a phase-contrast x-ray view.

In addition to the method according to at least one embodiment of the invention described above, the inventors also propose an x-ray CT system which has at least one emitter-detector system for simultaneous or temporally offset scanning and is suitable for creating at least one absorption x-ray CT view of a patient or of a region of a patient, and at least one phase-contrast x-ray CT view of the patient or of a second region of the patient, with an x-ray grating for generating quasi-coherent x-ray radiation being arranged between the x-ray source and the patient for at least measuring the phase-contrast, and with at least one further x-ray grating being arranged between the patient and the detector for determining the spatially dependent phase shift of the x-ray radiation in the patient, wherein a computational unit is provided which has a program memory in which a computer program code is stored which carries out the method steps of at least one embodiment of the method described above during the operation of the system.

According to the use described above of the method according to at least one embodiment of the invention for projective absorption records and projective phase-contrast records, the inventors also propose an x-ray projection system with at least one emitter-detector system for simultaneous or temporally offset scanning for creating at least one absorption x-ray projection view of a patient or of a region of a patient and at least one phase-contrast x-ray projection view of the patient or of a second region of the patient, with an x-ray grating for generating quasi-coherent x-ray radiation being arranged between the x-ray source and the patient for at least measuring the phase-contrast and at least one further x-ray grating being arranged between the patient and the detector for determining the spatially dependent phase shift of the x-ray radiation in the patient, wherein a computational unit is also intended to be provided in this case which has a program memory in which a computer program code is stored which at least carries out the method steps according to at least one embodiment of the invention described above during the operation of the system.

### BRIEF DESCRIPTION OF THE DRAWINGS

In the following, the invention will be described in more detail on the basis of the example embodiments with the aid of the figures, with only features required to understand the invention being illustrated. In this context, the following reference symbols are used: 1: x-ray phase-contrast CT system; 2: first x-ray tube; 2.1: first grating of the first tube-detector system; 3: first detector; 3.1: second grating of the first tube-detector system; 4: second x-ray tube; 4.1: first grating of the second tube-detector system; 5: second detector; 5.1: second grating of the second tube-detector system; 6: gantry housing; 7: patient; 8: patient couch; 9: system axis; 10: control and computational unit; 11: memory of the control and computational unit; 100-105, 200-207: method steps; AIₓ, AI_{y}: absorption views; PIₓ, PI_{y}: phase-contrast views; ΔPI_{xy}: phase-contrast x-ray difference view; Prg₁-Prgₓ: computer programs.

In particular,

FIG. 1 shows an x-ray phase-contrast CT system;

FIG. 2 shows a flowchart for direct functional imaging of a metabolic marker; and

FIG. 3 shows a flowchart for functional imaging of a metabolic marker by forming a difference.

### DETAILED DESCRIPTION OF THE EXAMPLE EMBODIMENTS

Various example embodiments will now be described more fully with reference to the accompanying drawings in which only some example embodiments are shown. Specific structural and functional details disclosed herein are merely representative for purposes of describing example embodiments. The present invention, however, may be embodied in many alternate forms and should not be construed as limited to only the example embodiments set forth herein.

Accordingly, while example embodiments of the invention are capable of various modifications and alternative forms, embodiments thereof are shown by way of example in the drawings and will herein be described in detail. It should be understood, however, that there is no intent to limit example embodiments of the present invention to the particular forms disclosed. On the contrary, example embodiments are to cover all modifications, equivalents, and alternatives falling within the scope of the invention. Like numbers refer to like elements throughout the description of the figures.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items.

It will be understood that when an element is referred to as being “connected,” or “coupled,” to another element, it can be directly connected or coupled to the other element or intervening elements may be present. In contrast, when an element is referred to as being “directly connected,” or “directly coupled,” to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

Although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

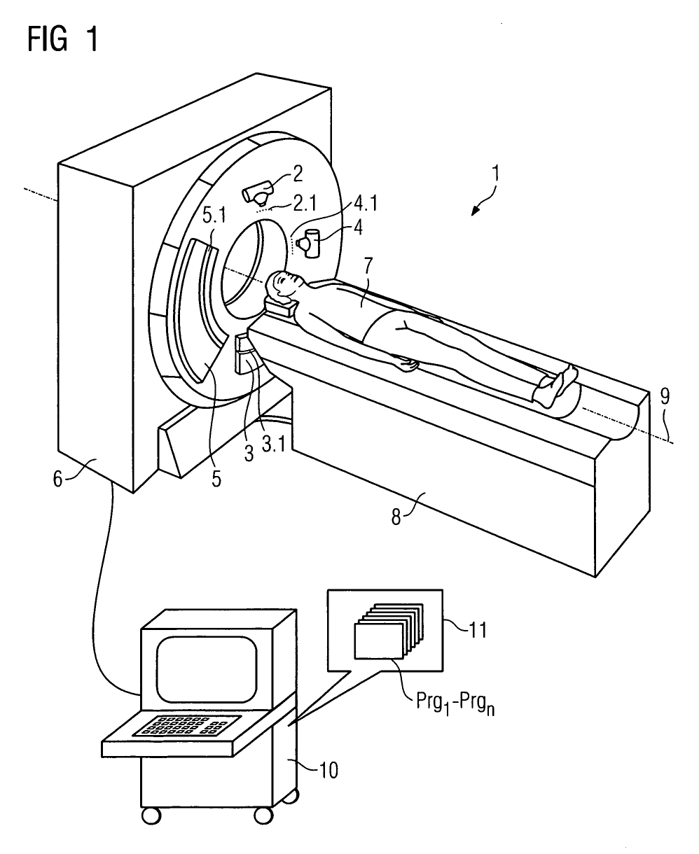

An example x-ray phase-contrast CT system 1 according to an example embodiment of the invention is shown in FIG. 1. It includes a gantry housing 6 with an interior gantry on which at least a first emitter-detector system, or optionally additional emitter-detector systems, is or are arranged. In this case, the first emitter-detector system comprises a first x-ray tube 2 with an x-ray absorption grating 2.1 arranged in front of the patient in order to create quasi-coherent x-ray radiation. Furthermore, the first emitter-detector system has a detector 3 opposite the x-ray tube 2 and with an x-ray grating 3.1 placed in front of it to “visualize” the phase shift of the x-ray radiation passing through the patient 7. For the purpose of the scan, the patient 7 is pushed along the system axis 9 though the measurement field on a patient couch 8, while the emitter-detector system on the gantry rotates about the system axis 9.

The basic method of operation and particular configuration of such phase-contrast CT systems has already repeatedly been explicitly described in various previous applications by the same applicant on the subject of phase-contrast CT and other publications in the prior art.

Optionally, the CT system can be equipped with one or more further emitter-detector systems as well. Here, a second emitter-detector system which can likewise rotate about the system axis 9 on the gantry and which has a second x-ray tube 4 with an absorption grating 4.1 and a detector 5 with a phase grating 5.1 placed in front of it is illustrated by way of example.

The CT system 1 is controlled by a control and computational unit 10, with this control and computational unit 10 keeping the work programs with their program code in a main memory 11 ready for operation. This main memory also contains, inter alia, programs Prg₁ to Prgₙ, which can emulate the previously described method according to an embodiment of the invention, and execute it during operation.

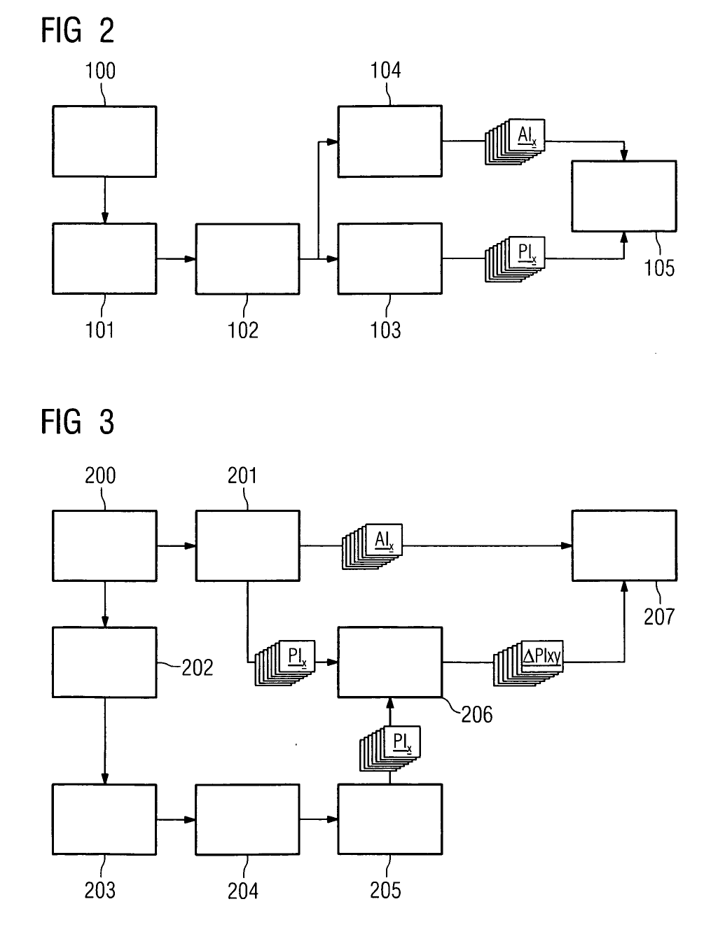

A specific method of an embodiment for detecting and localizing a metabolic marker in a patient is described in FIG. 2 with the aid of a flowchart, with boxes 100 to 105 representing the various method steps and the arrows between the boxes representing the order of working through the method steps.

In this specific method embodiment variant, a metabolic marker—a glucose solution for example—is initially administered to a patient in the step 100. According to method step 101, the reaching of a maximum accumulation of the marker at a location in the patient is now awaited. The waiting period can be estimated in this case. However, if the location of the observation in the patient is already known, it is also possible to carry out a plurality of test scans using a dose which is as low as possible during this waiting period, in order to observe the profile of the concentration of the marker. By way of example, if a known tumor focus is intended to be observed by administering glucose, the increase in the glucose concentration in the region of the tumor focus can be observed in the test scan, for example with a stationary and aligned x-ray tube but without the gantry being rotated and by only observing the projection data without reconstruction.

Once the actual or estimated concentration maximum of the marker has been reached, the actual data acquisition is carried out in step 102. Subsequently, in step 103, one or more phase-contrast CT views PIₓ and, in step 104, one or more absorption CT views AIₓ are reconstructed. With the aid of the absorption CT views AIₓ, the position of the patient in the CT, that is to say the anatomical information, can be determined in the best possible manner, while information about the more or less increased presence of the marker can be extracted from the phase-contrast CT views PIₓ. In step 105, these two sources of information are superposed on one another in a spatially precise manner, so that very precise medical information can be extracted from these combined displays. By way of example, the progressive development of a tumor, the metastasis or the reaction of a tumor to growth-inhibiting medicines can be observed, or the position of the tumor for radiation therapy planning can be determined very accurately. The options for use are extremely varied.

In another embodiment variant of the method according to an embodiment of the invention, it is proposed to observe the accumulation of metabolic markers after external administration by creating difference images from phase-contrast examinations. Such a procedure is illustrated, by way of example, in FIG. 3. In this case, boxes 200 to 207 describe the various method steps and the arrows between the boxes represent the order of working through the method steps.

This method begins at method step 200 with a first data acquisition by a first scan of the patient without applying a marker. From this first data acquisition, absorption x-ray CT and phase-contrast x-ray CT views AIₓ and PIₓ, respectively, are firstly calculated in step 201. This is subsequently followed by the administration of a bolus of a non-radioactive marker in step 202, and waiting for the increase of the marker concentration at specific locations in the patient in step 203. Optional test scans can also be carried out in this case. After the waiting period, a second data acquisition is carried out in step 204 and a second set of absorption x-ray CT and phase-contrast x-ray CT views AI_{y} and PI_{y}, respectively, are reconstructed in step 205, with the reconstruction of the absorption view being optional.

To avoid or reduce movement artifacts, these views can optionally be registered to the patient coordinate system of the first acquisition AIₓ with the aid of the absorption views AI_{y}.

Subsequently, the differences between the two phase-contrast views are calculated in step 206 by forming a pixel by pixel difference, that is to say a difference view ΔPI_{xy} is created, for improved accentuation of the differences. Finally, an absorption view AIₓ is superposed on this created phase-contrast difference view ΔPI_{xy} in step 207 and can, of course, be output on an appropriate display apparatus. In this case, optical accentuations can also be effected as a false-color display.

By way of this method, even relatively small differences in the metabolism can be recognized and anatomically associated in a very precise manner.

After studying this description, it is natural for a person skilled in the art to also apply the examples described in the scope of CT imaging to projection imaging.

It is self-evident that the previously mentioned features of the invention can be used not only in the respectively stated combination but also in other combinations or on their own, without departing from the scope of the invention.

Further, elements and/or features of different example embodiments may be combined with each other and/or substituted for each other within the scope of this disclosure and appended claims.

Still further, any one of the above-described and other example features of the present invention may be embodied in the form of an apparatus, method, system, computer program and computer program product. For example, of the aforementioned methods may be embodied in the form of a system or device, including, but not limited to, any of the structure for performing the methodology illustrated in the drawings.

Even further, any of the aforementioned methods may be embodied in the form of a program. The program may be stored on a computer readable media and is adapted to perform any one of the aforementioned methods when run on a computer device (a device including a processor). Thus, the storage medium or computer readable medium, is adapted to store information and is adapted to interact with a data processing facility or computer device to perform the method of any of the above mentioned embodiments.

The storage medium may be a built-in medium installed inside a computer device main body or a removable medium arranged so that it can be separated from the computer device main body. Examples of the built-in medium include, but are not limited to, rewriteable non-volatile memories, such as ROMs and flash memories, and hard disks. Examples of the removable medium include, but are not limited to, optical storage media such as CD-ROMs and DVDs; magneto-optical storage media, such as MOs; magnetism storage media, including but not limited to floppy disks (trademark), cassette tapes, and removable hard disks; media with a built-in rewriteable non-volatile memory, including but not limited to memory cards; and media with a built-in ROM, including but not limited to ROM cassettes; etc. Furthermore, various information regarding stored images, for example, property information, may be stored in any other form, or it may be provided in other ways.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A method for detecting and localizing a metabolic marker, the method comprising:
creating at least one absorption x-ray view of at least one of a patient and a first region of a patient;
creating at least one phase-contrast x-ray view of at least one of the patient and a second region of the patient, with a quasi-coherent x-ray radiation being generated for the phase-contrast measurement with the aid of an x-ray grating arranged between the x-ray source and the patient, and the spatially dependent phase shift of the x-ray radiation in the patient being made visible with the aid of at least one grating between the patient and a detector;
carrying out orientation based on anatomical features with the aid of the created at least one absorption x-ray view; and
determining a spatial distribution of the metabolic marker present in the body of the patient by the created at least one phase-contrast x-ray view.

2. The method as claimed in claim 1, wherein the patient is administered the metabolic marker to be localized, prior to the creation of at least one absorption x-ray view and at least one phase-contrast x-ray view.

3. The method as claimed in claim 1, wherein a first scan to create at least a first absorption x-ray view and at least a first phase-contrast x-ray view is carried out prior to the dose of the metabolic marker being administered and, after the dose of the metabolic marker has been administered, a second scan to create at least a second phase-contrast x-ray view is carried out.

4. The method as claimed in claim 3, wherein the second scan is also carried out to create a second absorption x-ray view.

5. The method as claimed in claim 3, wherein a difference image between the first and second phase-contrast x-ray view is created and displayed in order to accentuate the applied metabolic marker.

6. The method as claimed in claim 5, wherein one of the absorption x-ray views is superposed on the difference image.

7. The method as claimed in claim 4, wherein a spatial correction of the first and second phase-contrast x-ray views is carried out by registering the first phase-contrast x-ray view to the first absorption x-ray view and by registering the second phase-contrast x-ray view to the second absorption x-ray view, and by subsequently using the two spatially corrected phase-contrast x-ray views to create a spatially corrected difference image, with the difference image being used to accentuate the applied metabolic marker.

8. The method as claimed in claim 7, wherein a spatially corrected absorption x-ray view is superposed on the spatially corrected difference image.

9. The method as claimed in claim 1, wherein the phase-contrast x-ray view and the absorption x-ray view are displayed in different colors.

10. The method as claimed in claim 5, wherein the difference image and the absorption x-ray view are displayed in different colors.

11. The method as claimed in claim 3, wherein a period of time is allowed to elapse between the metabolic marker being administered and the second scan before an expected maximal marker concentration is reached.

12. The method as claimed in claim 3, wherein interim scans with a reduced dose are carried out between the metabolic marker being administered and the second scan, which scans reveal when a maximal maker concentration in a region of interest has been reached.

13. The method as claimed in claim 1, wherein the first and the second region are identical.

14. The method as claimed in claim 1, wherein the first region has a larger extent than the second region.

15. The method as claimed in claim 1, wherein glucose is used as a metabolic marker.

16. The method as claimed in claim 15, wherein the occurrence of an increased concentration of glucose or an increased concentration of glucose-6-phosphate is displayed.

17. The method as claimed in claim 1, wherein NaCl (common salt) is used as a metabolic marker.

18. The method as claimed in claim 1, wherein an absorption x-ray CT view is reconstructed and used as an absorption x-ray view, and a phase-contrast x-ray CT view is reconstructed and used as a phase-contrast x-ray view.

19. The method as claimed in claim 1, wherein an absorption x-ray projection view is used as an absorption x-ray view, and a phase-contrast x-ray projection view is used as a phase-contrast x-ray view.

20. An x-ray CT system, comprising:
an x-ray source;
at least one emitter-detector system for at least one of simultaneously and temporally offset scanning to create at least one absorption x-ray CT view of at least one of a patient and a region of a patient, and to create at least one phase-contrast x-ray CT view of at least one of the patient and a second region of the patient;
an x-ray grating for generating quasi-coherent x-ray radiation, being arranged between the x-ray source and the patient, to at least measure the phase-contrast;
at least one further x-ray grating, being arranged between the patient and the detector, to determine a spatially dependent phase shift of the x-ray radiation in the patient; and
a computational unit, including a program memory in which a computer program code is stored, to carry out the method of claim 1 during operation of the CT system.

21. An x-ray projection system, comprising:
an x-ray source;
at least one emitter-detector system for at least one of simultaneously and temporally offset scanning to create at least one absorption x-ray projection view of a patient and a region of a patient, and to create at least one phase-contrast x-ray projection view of at least one of the patient and a second region of the patient;
an x-ray grating for generating quasi-coherent x-ray radiation, being arranged between the x-ray source and the patient, to at least measure the phase-contrast;
at least one further x-ray grating, being arranged between the patient and the detector, to determine a spatially dependent phase shift of the x-ray radiation in the patient; and
a computational unit, including a program memory in which a computer program code is stored, to carry out the method of claim 1 during operation of the CT system.

22. The method as claimed in claim 1, further comprising superposing the created at least one absorption x-ray view and the created at least one phase-contrast x-ray view.

23. The method as claimed in claim 2, wherein a first scan to create at least a first absorption x-ray view and at least a first phase-contrast x-ray view is carried out prior to the dose of the metabolic marker being administered and, after the dose of the metabolic marker has been administered, a second scan to create at least a second phase-contrast x-ray view is carried out.

24. The method as claimed in claim 4, wherein a difference image between the first and second phase-contrast x-ray view is created and displayed in order to accentuate the applied metabolic marker.

25. At least one of x-ray CT system and an x-ray projection system, comprising:
means for creating at least one absorption x-ray view of at least one of a patient and a first region of a patient;
means for creating at least one phase-contrast x-ray view of at least one of the patient and a second region of the patient, with a quasi-coherent x-ray radiation being generated for the phase-contrast measurement with the aid of an x-ray grating arranged between the x-ray source and the patient, and the spatially dependent phase shift of the x-ray radiation in the patient being made visible with the aid of at least one grating between the patient and a detector;
means for carrying out orientation based on anatomical features with the aid of the created at least one absorption x-ray view; and
means for determining a spatial distribution of the metabolic marker present in the body of the patient by the created at least one phase-contrast x-ray view.

26. A computer readable medium including program segments for, when executed on a computer device, causing the computer device to implement the method of claim 1.
